(19) United States
(12) Patent Application Publication
(10) Pub. No.: US 2002/0197688 A1
(43) Pub. Date: Dec. 26, 2002

(54) Tobacco biomass utilization

(76) Inventor: Joseph Pandolfino, Clarence, NY (US)

Correspondence Address: PHILLIPS, LYTLE, HITCHCOCK, BLAINE & HUBER LLP, 3400 HSBC CENTER, BUFFALO, NY, 14203-3509, US

(21) Appl. No.: 10/163,101

(22) Filed: Jun. 5, 2002

Related U.S. Application Data
(60) Provisional application No. 60/296,347, filed on Jun. 6, 2001.

(51) Int. Cl. C12P 7/06; A01H 5/00

(52) U.S. Cl. 435/161; 800/284

(57) ABSTRACT

The present invention discloses a method for the fermentative production of ethanol comprising providing a reduced nicotine recombinant tobacco plant or a portion thereof, fermenting the plant or portion thereof in a fermentation vessel for a time sufficient to produce ethanol therefrom, and then collecting the ethanol from the fermentation vessel. The present invention also discloses a method of sustaining an animal subject comprising feeding the animal subject a reduced nicotine recombinant tobacco plant or a portion thereof. The present invention also discloses a method of producing a protein fraction from plant biomass comprising providing a reduced nicotine recombinant tobacco plant or a portion thereof and then collecting a protein fraction from the recombinant plant or plant portion.

### FIELD OF THE INVENTION

The present invention provides methods for the use of reduced nicotine tobacco as, among other things, sources of protein, fiber, ethanol, and animal feeds.

### BACKGROUND OF THE INVENTION

Tobacco plants can be used to efficiently achieve large quantities of biomass. This biomass can be produced by simply direct-planting tobacco seeds close together in a field. Since tobacco vigorously regenerates itself when cut, a field planted with tobacco can be mowed when plants reach about eighteen to twenty-four inches in height (conventional tobacco plants are usually harvested at about four feet) and new, thick, dense growth will replace the mowed plants. If plants are harvested in this manner three to four times per growing season, approximately 100 tons of tobacco biomass can be produced per acre. After subtracting the eighty to ninety percent water, as most plants contain, about 10 to 20 tons of dry solid weight remains. In addition to being highly efficient at producing biomass, tobacco is an extremely versatile plant. Unlike grains such as wheat and corn, tobacco does not have a substantial amino acid deficiency. Tobacco is a substantially allergen-free source of protein, which protein contains all 20 amino acids important to humans and most livestock, and has a great proportion of digestible sugars and starches. Tobacco is also a superior source of dietary fiber and is more digestible than fescue hay by ruminant animals. Bulk fiber volume of tobacco is much higher than that of alfalfa and wheat bran. By growing tobacco biomass, good agricultural land can be diverted to raising other crops: an important consideration for countries or locations with limited farmland.

Accordingly, it would be desirable to provide new ways to utilize this plant in a productive, effective and non-toxic manner.

### SUMMARY OF THE INVENTION

The present invention is based upon the finding that reduced-nicotine recombinant tobacco is well-suited to a variety of alternative uses, including but not limited to: (a) as forage crop for livestock and the like; (b) as an animal feed or feed ingredient; (c) as a source of proteins; (d) as a source of fiber for incorporation into animal feeds and food products; (e) a substrate for the fermentative production of ethanol; (f) as a source of recombinant or transgenic proteins produced through “molecular farming” techniques; and (g) in combination processes implementing two or more of the foregoing based upon a single plant, or batch or crop of plants.

In general, reduced nicotine recombinant tobacco plants used to carry out the present invention contain and express a heterologous DNA encoding at least a segment of an enzyme required for the biosynthesis of nicotine in tobacco, the plant exhibiting reduced levels of the enzyme as compared to a non-transformed control plant and reduced nicotine content as compared to a non-transformed control plant. In a preferred embodiment, the plants further exhibit increased levels of reducing sugars as compared to a non-transformed control plant.

A first aspect of the present invention is a method of producing ethanol, comprising: providing a reduced nicotine recombinant tobacco plant as described above; fermenting the plant in a fermentation vessel for a time sufficient to produce ethanol therefrom; and then collecting the ethanol from the fermentation vessel. The term “plant” includes physical and chemical portions thereof, such as plant parts and plant extracts, hydrolysates, etc.

A second aspect of the present invention is a method of sustaining an animal subject, comprising feeding the animal subject reduced nicotine recombinant tobacco as described above. Feeding may be carried out by any suitable means, such as by feeding the animal a forage crop comprising the tobacco; by feeding the animal leaves from the tobacco; by feeding the animal silage comprising the tobacco; and/or by feeding the animal a food product comprising the tobacco, optionally in combination with at least one additional nutrient.

A third aspect of the present invention is a food product comprising a reduced nicotine recombinant tobacco plant. The food product may take any suitable form, such as silage or the tobacco plant in combination with at least one additional nutrient. The food product may comprise leaves (that is, discernible leaves or leaf fragments or pieces from the plant) or other fractions isolated from the plant.

A fourth aspect of the present invention is a method of producing a protein fraction (e.g., fraction I protein and/or fraction II protein) from plant biomass, comprising the steps of: providing reduced nicotine recombinant tobacco plants as described above, and then collecting a protein fraction from the recombinant tobacco plants.

A still further aspect of the present invention involves the use of recombinant tobacco plants as described above for molecular farming.

The foregoing and other objects and aspects of the present invention are explained in greater detail in the specification set forth below.

### DETAILED DESCRIPTION OF THE PREFERRED EMBODIMENTS

Plants for use in the present methods are species of the genus Nicotiana, or tobacco, including without limitation N tabacum, N rustica and N glutinosa. As used herein, reference to “tobacco” means and encompasses any plant, species, or hybrids of the genus Nicotiana. Any strain or variety of tobacco may be used. Preferred are strains that are already low in nicotine content, such as Nic1/Nic2 double mutants. The plants are modified to reduce the nicotine content thereof by transgenic means as discussed in greater detail below.

As used herein, reference to “reduced nicotine” means a recombinant (or “transgenic”) tobacco plant that contains less than half, preferably less than 25%, and more preferably less than 20% or less than 10% of the nicotine content of the non-transgenic “parent” (or unmodified “control”) plant from which the transgenic plant is produced. It will be appreciated that some small level of residual nicotine, on the order of at least 1% or 5% as compared to the corresponding unmodified control plant, may remain in the transgenic plants used to carry out the instant invention.

It is specifically intended that the disclosure of all United States patent references cited herein be incorporated by reference herein in their entirety.

A. Reduced-Nicotine Tobacco Plants.

Reduced-nicotine tobacco plants used to carry out the instant invention are, in general, recombinant tobacco plants that contains and express a heterologous nucleotide, the expression of which heterologous nucleotide downregulates an enzyme such as quinolate phosphoribosyl transferase (QPRTase), putrescene methyl transferase (PMTase), arginine decarboxylase, omithine decarboxylase, S-adenosylmethionine synthetase, NADH dehydrogenase, or phosphoribosylanthranilate isomerase in the plant, and thereby reduces the production of nicotine in the plant. Suitable recombinant plants are disclosed in M. Conkling et al., PCT Application WO98/56923 (published Dec. 17, 1998) and in M. Timko, PCT Application WO00/67558 (published Nov. 16, 2000), the disclosures of which are incorporated herein by reference. In general, the heterologous nucleotide comprises at least a segment of a nucleic acid encoding the enzyme to be down-regulated, in sense or antisense orientation.

Preferably the reduced nicotine tobacco also contains reduced (e.g., by at least 90, 95 or 99 percent by weight or more) levels of tobacco-specific nitrosamines as compared to that which would be found in the plant in the absence of corresponding reductions in nicotine.

One embodiment of the present invention utilizes a reduced nicotine recombinant plant that has reduced quinolate phosphoribosyl transferase (QPRTase) expression relative to a non-transformed control plant, said recombinant plant comprising recombinant plant cells containing: an exogenous DNA construct comprising, in the 5′ to 3′ direction, a promoter operable in said plant cell and a heterologous DNA encoding at least a segment of a plant quinolate phosphoribosyl transferase mRNA, said heterologous DNA operably associated with said promoter, and with said heterologous DNA in sense or antisense orientation; said plant exhibiting reduced QPRTase expression compared to a non-transformed control plant and reduced nicotine content as compared to a non-transformed control plant.

Another embodiment of the invention may be carried out with a reduced nicotine recombinant plant that has reduced putrescine N-methyltransferase (PMTase) expression relative to a non-transformed control plant, said recombinant plant comprising recombinant plant cells containing: an exogenous DNA construct comprising, in the 5′ to 3′ direction, a promoter operable in said plant cell and a heterologous DNA encoding at least a segment of a plant PMT mRNA, said heterologous DNA operably associated with said promoter, and with said heterologous DNA in sense or antisense orientation; said plant exhibiting reduced PMT expression compared to a non-transformed control plant and reduced nicotine content as compared to a non-transformed control plant. Still other embodiments may be carried out in like manner with the other enzymes listed above.

Nucleic acid constructs as described above may include insulator elements upstream (5′ to) and/or downstream (3′ to) of the construct described above, as set forth (for example) in U.S. Pat. Nos. 6,100,448 and 6,037,525 to Thompson et al. In addition, constructs as described above may include matrix (or scaffold) attachment regions upstream and/or downstream of the construct described above, as set forth (for example) in U.S. Pat. Nos. 5,773,695 and 5,773,689 to Thompson et al.

While plants described herein are generally described as possessing a single recombinant nucleic acid that down-regulates a single enzyme in the nicotine synthesis pathway, it will be appreciated that plants utilized in the invention may contain a plurality of recombinant nucleic acids that down-regulate a plurality of enzymes in the nicotine synthesis pathway (e.g., both PMTase and QPRTase). Plants described as possessing a single recombinant nucleic acid may thus encompass those containing the plurality.

Thus, still another embodiment of the present invention utilizes a reduced nicotine recombinant plant that has both reduced QPRTase and reduced PMTase expression relative to a non-transformed control plant, said recombinant plant comprising recombinant plant cells containing: (i) a first exogenous DNA construct comprising, in the 5′ to 3′ direction, a promoter operable in said plant cell and a heterologous DNA encoding at least a segment of a plant quinolate phosphoribosyl transferase mRNA, said heterologous DNA operably associated with said promoter; and (ii) a second exogenous DNA construct comprising, in the 5′ to 3′ direction, a promoter operable in said plant cell and a heterologous DNA encoding at least a segment of a plant PMT mRNA, said heterologous DNA operably associated with said promoter, and with said heterologous DNA in sense or antisense orientation; said plant exhibiting reduced PMT expression compared to a non-transformed control plant and reduced nicotine content as compared to a non-transformed control plant. It will be appreciated that, where sense and antisense downregulation are described herein, other techniques such as the use of ribozymes or interfering complementary mRNA may be used.

Examples of nucleic acid sequences that may be used to carry out the present invention include, but are not limited to, DNA encoding the tobacco quinolate phosphoribosyl transferase gene (NtQPT1), which is known (see, e.g., PCT Application WO98/5556923 to Conkling et al.; and DNA encoding tobacco putrescine N-methyltransferase such as PMT1, PMT2, PMT3 and PMT4), DNA encoding tobacco arginine decarboxylase such as ADC1 and ADC2, DNA encoding tobacco omithine decarboxylase (ODC), DNA encoding tobacco S-adenosylmethionine synthetase (SAMS), DNA encoding tobacco NADH dehydrogenase, and DNA encoding tobacco phosphoribosylanthranilate isomerase (which are known and described in PCT Application WO 00/67558 to M. Timko et al.).

Conditions which permit other DNA sequences which code for expression of a protein having a desired enzyme activity as described above to hybridize to DNA as described above, or to other DNA sequences encoding the enzyme protein as given above, can be determined in a routine manner. For example, hybridization of such sequences may be carried out under conditions of reduced stringency or even stringent conditions (e.g., conditions represented by a wash stringency of 0.3 M NaCl, 0.03 M sodium citrate, 0.1% SDS at 60° C. or even 70° C. to DNA encoding the protein given above in a standard in situ hybridization assay. See J. Sambrook et al., Molecular Cloning, A Laboratory Manual (2d Ed. 1989)(Cold Spring Harbor Laboratory)). In general, such sequences will be at least 65% similar, 75% similar, 80% similar, 85% similar, 90% similar, or even 95% similar, or more, with the sequence given above, or DNA sequences encoding proteins given above. (Determinations of sequence similarity are made with the two sequences aligned for maximum matching; gaps in either of the two sequences being matched are allowed in maximizing matching. Gap lengths of 10 or less are preferred, gap lengths of 5 or less are more preferred, and gap lengths of 2 or less still more preferred.)

The heterologous sequence utilized in the methods of the present invention may be selected so as to produce an RNA product complementary to the entire message encoding the enzyme sequence, or to a portion thereof. The sequence may be complementary to any contiguous sequence of the natural messenger RNA, that is, it may be complementary to the endogenous mRNA sequence proximal to the 5′-terminus or capping site, downstream from the capping site, between the capping site and the initiation codon and may cover all or only a portion of the non-coding region, may bridge the non-coding and coding region, be complementary to all or part of the coding region, complementary to the 3′-terminus of the coding region, or complementary to the 3′-untranslated region of the mRNA. Suitable antisense sequences may be from at least about 12, 14 or 15 to about 15, 25, or 35 nucleotides, at least about 50 nucleotides, at least about 75 nucleotides, at least about 100 nucleotides, at least about 125 nucleotides, at least about 150 nucleotides, at least about 200 nucleotides, or more. In addition, the sequences may be extended or shortened on the 3′ or 5′ ends thereof (e.g., by the addition of 1 to 4 or 8 additional nucleic acid residues). The antisense product may be complementary to coding or non-coding (or both) portions of naturally occurring target RNA. The particular anti-sense sequence and the length of the anti-sense sequence will vary depending upon the degree of inhibition desired, the stability of the anti-sense sequence, and the like. One of skill in the art will be guided in the selection of appropriate enzyme antisense sequences using techniques available in the art and the information provided herein.

As indicated above, the present invention may also be carried out with plants that implement sense co-suppression of nicotine production. Sense DNAs employed in carrying out the present invention are of a length sufficient to, when expressed in a plant cell, suppress the native expression of the plant enzyme as described herein in that plant cell. Such sense DNAs may be essentially an entire genomic or complementary DNA encoding the enzyme, or a fragment thereof with such fragments typically being at least 15 nucleotides in length. Methods of ascertaining the length of sense DNA that results in suppression of the expression of a native gene in a cell are available to those skilled in the art. The present invention may also be carried out with plants that contain DNAs encoding double stranded RNAs comprising complementary antisense and sense sequences that when expressed are capable of suppressing or silencing endogenous genes containing the sequences. Suitable complementary regions may be from at least about 20 to 25 nucleotides and may be separated by at least about 5 nucleotides.

In still another embodiment of the present invention, Nicotiana plant cells are transformed with a DNA construct containing a DNA segment encoding an enzymatic RNA molecule (i.e., a “ribozyme”), which enzymatic RNA molecule is directed against (i.e., cleaves) the mRNA transcript of DNA encoding a plant enzyme as described herein. Ribozymes contain substrate binding domains that bind to accessible regions of the target mRNA, and domains that catalyze the cleavage of RNA, preventing translation and protein production. The binding domains may comprise antisense sequences complementary to the target mRNA sequence; the catalytic motif may be a hammerhead motif or other motifs, such as the hairpin motif. Ribozyme cleavage sites within an RNA target may initially be identified by scanning the target molecule for ribozyme cleavage sites (e.g., GUA, GUU or GUC sequences). Once identified, short RNA sequences of 15, 20, 30 or more ribonucleotides corresponding to the region of the target gene containing the cleavage site may be evaluated for predicted structural features. The suitability of candidate targets may also be evaluated by testing their accessibility to hybridization with complimentary oligonucleotides, using ribonuclease protection assays as are known in the art. DNA encoding enzymatic RNA molecules may be produced in accordance with known techniques. See, e.g., T. Cech et al., US. Patent No. 4,987,071;Donson et al., U.S. Pat. No. 5,589,367; Torrence et al., U.S. Pat. No. 5,583,032; Joyce, U.S. Pat. No. 5,580,967; Wagner et al., U.S. Pat. No. 5,591,601; and U.S. Pat. No. 5,622,854. Production of such an enzymatic RNA molecule in a plant cell and disruption of enzyme protein production reduces enzyme activity in plant cells in essentially the same manner as production of an antisense RNA molecule: that is, by disrupting translation of mRNA in the cell which produces the enzyme. The term ‘ribozyme’ is used herein to describe an RNA-containing nucleic acid that functions as an enzyme (such as an endoribonuclease), and may be used interchangeably with ‘enzymatic RNA molecule’.

In still another embodiment of the invention, down-regulation of nicotine production may be achieved by employing translational inhibition of mRNA utilizing interfering complementary mRNA, as set forth in U.S. Pat. No. 5,272,065.

To produce a tobacco plant having decreased enzyme levels, and thus lower nicotine content, than an untransformed control tobacco plant, a tobacco cell may be transformed with an exogenous transcriptional unit comprising a partial enzyme nucleic acid sequence, a full-length enzyme nucleic acid sequence, in the sense or antisense orientation with appropriate operably linked regulatory sequences or a sequence encoding a ribozyme as described above. Appropriate regulatory sequences include a transcription initiation sequence (“promoter”) operable in the plant being transformed, and a polyadenylation/transcription termination sequence. Standard techniques, such as restriction mapping, Southern blot hybridization, and nucleotide sequence analysis, are then employed to identify clones bearing enzyme sequences in the antisense orientation, operably linked to the regulatory sequences. Tobacco plants are then regenerated from successfully transformed cells. It is most preferred that the antisense sequence utilized be complementary to the endogenous sequence, however, minor variations in the exogenous and endogenous sequences may be tolerated. It is preferred that the antisense DNA sequence be of sufficient sequence similarity that it is capable of binding to the endogenous sequence in the cell to be regulated, under stringent conditions as described below. Particular techniques for producing recombinant tobacco plants are known to those skilled in the art and are explained in greater length in M. Conkling et al., PCT Application WO98/56923 (published Dec. 17, 1998) and in M. Timko, PCT Application WO00/67558 (published Nov. 16, 2000), noted above.

B. Tobacco Leaf as an Animal Feed or Food Supplement.

A second aspect of the present invention is a method of sustaining an animal subject, comprising feeding the animal subject reduced nicotine recombinant tobacco as described above (including both plants and plant portions). Animals that may be fed with plants in accordance with the present invention (or food products as described below) include mammals (including ruminants), such as cows, sheep, and pigs, as well as poultry such as chickens and turkeys, fish, and others.

In one embodiment, the plants or plant portions used as food, or to produce food products, are those containing elevated levels of reducing sugars as described above.

Feeding of animals may be carried out by any suitable means, such as by feeding the animal a forage crop comprising the tobacco, by feeding the animal leaves from the tobacco; by feeding the animal silage comprising the tobacco; and/or by feeding the animal a food product comprising the tobacco in combination with at least one additional nutrient.

A food product of the present invention comprises a reduced nicotine recombinant tobacco plant. The plant may be included in the food product in any suitable form, such as leaves or chopped leaves, plant portions (e.g., extracts containing reducing sugars), etc. The food product may comprise any suitable amount of the recombinant plant (or plant portion), for example from 1, 5 or 10 percent to 90, 95 or 99 percent by weight (or more), with the balance of the product comprising other nutrients or additives. The food product may take any suitable form, such as silage or the tobacco plant in combination with at least one additional nutrient. The food product may comprise leaves (that is, discernible leaves or leaf fragments or pieces from the plant) or other fractions isolated from the plant. The food product may be dried or freeze-dried if desired. When raised as a forage crop, the tobacco plants may be simply fed to the animal from stalks as planted in the ground, or dried or partially dried and baled for subsequent use, and may optionally be treated with other ingredients (see, e.g., U.S. Pat. No. 4,034,117 to Glabe), or stored and at least partially fermented and used as silage (see, e.g., U.S. Pat. No. 4,015,018 to Glabe et al.).

Tobacco plants used as animal food or to produce food products in accordance with the present invention may be genetically engineered to contain and express a heterologous nucleic acid encoding phytase so that elevated levels of phytase are contained in the plants, as compared to an unmodified control plant, as described in U.S. Pat. No. 5,900,525 to S. Austin-Phillips et al. (WARF).

Other nutrients or additives that can be combined with plants or plant portions as described herein to produce a food product of the invention may include carbohydrates fats, lipids, and/or proteins. Carbohydrate sources used to produce an animal feed according to the present invention include, for example, corn, oats, barley, sorghum, or combinations of the same. These grains are preferably ground into a meal for use in the animal feed. Supplementary protein sources include, for example, soy meal, fish meal, blood meal, poultry by-product (ground poultry offal), meat meal, feather-lysate (see, e.g., U.S. Pat. No. 4,908,220 to Shih) and combinations of the same. An animal feed is comprised of from about 13% to about 25 percent by weight of protein from all protein sources (both from the tobacco plants and others). The plant or plant portions may be the sole protein source, or supplemented as above. Other nutrients in small amounts, such as vitamins, minerals, antibiotics, and other substances or compounds may be included in the feed as required. The ingredients may be mixed and blended in accordance with any suitable procedure and formulated into any suitable form, such as food pellets. The tobacco can be transgenically modified to provide for the production of additional nutrients therein, if desired.

C. Ethanol Fermentations with Tobacco.

Fermentation processes in which ethanol or other materials such as acetone are produced from plant material are well known in the literature. See, e.g., C. Weizmann, Production of Acetone and Alcohol by Bacteriological Processes, U.S. Pat. No. 1,315,585 (1919). The desired product may be produced from fermentation of constituents such as sugars in the biomass, from cellulose and hemicellulose in the biomass, or both. In general sugars and starches are simpler to ferment than cellulose and hemicellulose. The fermentation process can be embodied in any of a variety of forms, a few examples of which are given below. Note that fermentation can be carried out on raw plant material or various constituents of the plant material (e.g., sugar and cellulose fractions), the latter being preferred when other constituents such as protein fractions are separated from the plant material for other purposes, such as isolation of fraction I and/or fraction II proteins as described below. Also, the biomass may be pre-processed to render it suitable for particular fermentation processes, depending upon the process employed (e.g., enzymatic digestion of cellulose to produce soluble sugars).

One particular example of a fermentation process that may be used to carry out the present invention is provided in U.S. Pat. No. 4,663,284 to Jeffries, which discloses a process for producing ethanol from D-xylose by fermentation with xylose metabolizing yeasts, wherein small quantities of glucose are added to the fermentation medium during the fermentation process.

U.S. Pat. No. 4,511,656 to Gong provides a method for producing ethanol directly from D-xylose through fermentation of D-xylose by yeast mutants. The process further provides for directly and simultaneously obtaining ethanol from a mixture of cellulose and hemicellulose through yeast fermentation of D-glucose and D-xylose

U.S. Pat. No. 4,490,468 to Gong et al. provides an anaerobic fermentation of xylulose previously obtained by isomerization of xylose.

U.S. Pat. No. 4,368,268 to Gong provides a process for the production of ethanol from xylulose. The process includes isomerizing xylose to xylulose and fermenting the xylulose to ethanol. Essentially, this process is the fermentation of xylose, alone or in combination with other sugars, in hemicellulose hydrolysates by mutant strains of yeast, either aerobically or anaerobically.

U.S. Pat. No. 4,840,903 to Wu provides a process for producing ethanol from plant biomass. The process includes forming a substrate from the biomass, with the substrate including hydrolysates of cellulose and hemicellulose. A species of the fungus Paecilomyces which has the ability to ferment both cellobiose and xylose to ethanol (e.g., Paecilomyces sp. NF1) is selected and isolated, and the substrate is then inoculated with this fungus. The inoculated solution is fermented under conditions favorable for cell viability and conversion of hydrolysates to ethanol, and the ethanol is recovered from the fermented solution.

U.S. Pat. No. 5,100,791 to D. Spindler et al. describes a process for producing ethanol from plant biomass. The process includes forming a substrate from the biomass with the substrate including hydrolysates of cellulose and hemicellulose. A species of the yeast Brettanomyces custersii (CBS 5512), which has the ability to ferment both cellobiose and glucose to ethanol, is then selected and isolated. The substrate is inoculated with this yeast, and the inoculated substrate is then fermented under conditions favorable for cell viability and conversion of hydrolysates to ethanol.

U.S. Pat. No. 5,372,939 to M. Lastick et al. describes a process for producing ethanol from a mixed stream of xylose and cellulose using enzymes to convert these carbohydrates to fermentable sugars under predetermined conditions. This is done by the simultaneous conversion of cellulose to glucose, using cellulase enzymes, and the conversion of xylose to xylulose, using the enzyme xylose isomerase in the presence of Schizosaccharomyces pombe ATCC No. 2476. The enzymatic processes allow for these fermentable sugars, glucose and xylulose, to be converted by yeast to ethanol in the same fermentation.

The foregoing are illustrative of fermentation processes that may be used to carry out the present invention, and are not intended to be limiting. Numerous other processes will be readily apparent to those skilled in the art and may be used to carry out the fermentations described herein. Also, while fermentation processes are herein described as primarily for the production of ethanol, it will be appreciated that other solvents such as acetone may be produced by fermentations of the invention as well.

D. Separation of Fraction I and Fraction II Proteins from Tobacco Leaf.

As described in U.S. Pat. Nos. 4,347,324 and 4,268,632 to S. Wildman and P. Kwanyuen (Leaf Proteins Inc.), the succulent leaves of certain plants, including tobacco, are composed of 10-20% solids, the balance being water. The solid portion is composed of a water soluble portion and a water insoluble portion, the latter being made up, for the most part, of the fibrous structural material of the leaf.

The water soluble portion is divisible into two groups. One group includes compounds of relatively lower molecular weight. The second group is almost exclusively proteins whose molecular weights are about 30,000 daltons or greater. The proteins of the second group can be resolved into two fractions. One fraction contains a mixture of proteins whose molecular weights range from about 30,000 daltons to 100,000 daltons. These proteins are sometimes referred to as “Fraction 2 proteins.” The remaining fraction comprises a single protein having a molecular weight of about 550,000 daltons and is sometimes referred to as “Fraction 1 protein.”

Fraction 1 protein is an enzyme involved in photosynthesis and is also known as ribulose 1,5-diphosphate carboxylase, carboxydismutase, ribulose 1,5-bisphosphate carboxylase and ribulose 1,5-di(or bis) phosphate carboxylase-oxygenase. Fraction 1 protein may compose up to 25% of the total protein content of a leaf and up to 10% of the solid matter in the leaf.

Fraction 1 protein, when pure, is odorless, tasteless and colorless and has high nutritional value. In view of these properties, and because it can be obtained in high purity, Fraction 1 protein is considered to have a potentially valuable application as a food supplement for animals and humans. In the case of humans, the additive could be a component of high protein or other special diets. It has, for example, been suggested as a supplement to the diet of persons who require dialysis because of kidney disease.

A variety of processes for isolating Fraction 1 protein are known. Three basic processes for isolating Fraction 1 protein are described in U.S. Pat. Nos. 4,347,324 w and 4,268,632 to S. Wildman and P. Kwanyuen. Each of these three methods begins with pulping the leaves, or leaves and stalk of the plant, followed by expressing a green juice from the pulp. The green juice, which contains finely particulate green pigmented material, is clarified for example, by filtration or centrifugation, to separate the finely particulate solid matter from the liquid. The resulting liquid is brown in color.

The first method involves concentration of Fraction 1 protein simultaneously with its partial separation from lower molecular weight compounds in the brown juice by molecular filtration. Using a molecular sieve whose pores would pass smaller molecules without passing Fraction 1 protein, the brown juice is placed under pressure so that small molecules would pass through the pores. The solution containing the Fraction 1 protein is then concentrated about ten-fold and then dialyzed to remove additional small molecules in the solution. Dialysis is accomplished using a collodion type dialysis bag. The pores of the bag do not permit passage of the Fraction 1 protein but allow the smaller molecules to escape through the bag into water. During dialysis, crystals of Fraction 1 protein formed.

The second method involves passing the brown juice obtained from the leaves through a Sephadex chromatographic column. Either Sephadex G-25 or G-50 may be used to perform the separation. Selection of proper beads permits small molecules to penetrate to the interior of their structure to the exclusion of larger molecules. The larger molecules, therefore, are only found in the liquid in the interstices between the tightly packed Sephadex beads. This interstitial space is referred to as the “void volume”. To achieve effective separation, the volume of brown juice cannot exceed about 25% of the total volume of the beads. The beads are first equilibrated with a buffer and a volume of brown juice, containing the same buffer, is then layered on top of the Sephadex column. The brown liquid is eluted from the column using the buffer solution. As the juice moves down the column, the passage of small molecules is retarded since they penetrate the interior of the beads. The large Fraction 1 molecules, on the other hand, move at a faster rate down the column through the labyrinth formed by the interstices between the beads and emerges from the column as a clear brown solution. However, elution results in at least two-fold dilution of the solution. Removal of the smaller molecules changes the environment around the molecules of Fraction 1 protein which leads to crystallization.

A third method involves passage of the brown juice through a Sephadex G-25 column as described above. If Fraction 1 protein does not crystallize, as is the case with the extract of all plants except tobacco, ammonium sulfate is added until the solution is 30-50% saturated. This leads to precipitation of an amorphous material which is collected by centrifugation. After separation, the precipitate is redissolved in a smaller volume of buffer than that from which it was precipitated to which is added 8% polyethylene glycol. This mixture is placed in an open dish adjacent to another open dish containing silica gel and the two dishes confined in a closed vessel. Water is gradually evaporated from the protein solution and absorbed by the silica gel. With the passage of time, crystals of Fraction 1 protein develop.

U.S. Pat. No. 4,268,632 to Wildman and Kwanyuen describes a process for isolating Fraction I protein comprising the steps of converting the leaves to a pulp, heating the liquid portion of the pulp to a temperature below that which causes the protein to denature followed by cooling the liquid portion to a temperature at which Fraction 1 protein, i.e., ribulose 1,5-diphosphate carboxylase crystallizes. U.S. Pat. No. 4,347,324 to Wildman and Kwanyuen describes an improvement to the foregoing in which the heating step originally believed to have been essential can be eliminated. By the improved process, ribulose 1,5-diphosphate carboxylase is obtained in crystalline form by adjusting the pH of the liquid portion of a pulp derived from the leaves to a value in the range of from between about 6 to a pH above that at which the protein will denature and precipitate as an amorphous mass, i.e., to a value above the isoelectric point which occurs at about pH 5.0. The liquid, after separation of insoluble material, is then permitted to stand, preferably while cooled below ambient temperature, to permit crystallization of the Fraction 1 protein.

U.S. Pat. No. 4,400,471 to D. Bourque (University Patents, Inc.) describes a method of crystallizing fraction I protein from a photosynthetic organism comprising: separating and purifying protein from the plant; mixing the protein in a suitable solvent to form a protein solution at a predetermined pH; mixing a precipitant solution with the protein solution, the precipitant solution having a pH lower than the pH of the protein solution and within the range of 4.8 to 7.2 to form a mixed solution having a pH in the range of 6.6 to 7.0; and then removing a portion of the solvent from the mixed solution to cause the protein to crystallize.

U.S. Pat. No. 4,400,471 to S. Johal describes a method for preparing ribulose 1,5-bisphosphate carboxylase (RuBisCO) from plant material such as tobacco leaves that comprises the steps of: comminuting the plant material in an aqueous solution to form a suspension; fractionating the suspension to release the RuBisCO from the ground plant material into the solution; adding a sufficient amount of polyethylene glycol (PEG) to the solution so that crystals of RuBisCO are selectively formed, said crystals having impurities; separating the crystals from the solution; redissolving the crystals in water; passing the redissolved crystals through an anionic resin bed; washing the column to remove the unbound material; and passing a single solution containing predetermined concentration of a divalent metal ion through the column at a concentration sufficient to selectively elute the RuBisCO from the resin. See also U.S. Pat. No. 4,588,691 to S. Johal.

Fraction II proteins can be separated in the course of separating fraction I proteins as the remaining soluble proteins after the fraction I proteins have been separated, or by other techniques which will be apparent from the aforesaid discussion of the separation of fraction I proteins.

E. Molecular Farming.

As noted in M. Conkling et al., PCT Application WO98/56923, Tobacco plants with low levels of nicotine production, or no nicotine production, are attractive as recipients for transgenes expressing commercially valuable products such as pharmaceuticals, cosmetic components, or food additives. Suitable techniques for molecular farming are described in, among other things, U.S. Pat. Nos. 6,096,547; 5,629,175; and 5,550,038, all to Goodman et al. (Calgene). Tobacco is attractive as a recipient plant for a transgene encoding a desirable product, as tobacco is easily genetically engineered and produces a very large biomass per acre; tobacco plants with reduced resources devoted to nicotine production accordingly will have more resources available for production of transgene products. Methods of transforming tobacco with transgenes producing desired products are known in the art; any suitable technique may be utilized with the low nicotine tobacco plants of the present invention.

Examples of desireable products that may be encoded by a transgene and expressed in tobacco plants as described above (including those tobacco plants described in PCT Application WO00/67558) include, but are not limited to, mammalian, particularly human, proteins and peptides such as Interleukin-1 (IL-1), IL-2, IL-3, IL-4, Il-5, IL-6, IL-7, Il-8, IL-9, IL-10, IL-11, IL-12 and other lymphokines, erythropoietin or EPO, interferons such as alpha, beta and gamma interferon, growth factors such as G-CSF, GM-CSF, and M-CSF, blood factors such as factors I to XII, particularly Factor VIII and Factor IX, tissue plasminogen activator or tPA, insulin-like growth factor, epidermal growth factor, platelet-derived growth factor, transforming growth factor-alpha, beta, etc., growth hormone, insulin, collagen plasminogen activator, histocompatibility antigens, receptors, receptor antagonists, antibodies, single-chain antibodies, enzymes, neuropolypeptides, antigens, vaccines, peptide hormones, calcitonin, human growth hormone, and phytase, as well as antimicrobial peptides or proteins such as protegrins, magainins, cecropins, cercosporins, melittins, indolicidins, defensions, β-defensins, cryptdins, clavainins, plant defensins, nicin, bactenecins, etc.

In like manner as for the constructs concerning enzymes in the nicotine synthetic pathway, the constructs encoding the aforesaid proteins and peptides inserted into the tobacco plant may include insulator elements upstream (5′ to) and/or downstream (3′ to) of the construct described above, as set forth (for example) in U.S. Pat. Nos. 6,100,448 and 6,037,525 to Thompson et al, or may include matrix (or scaffold) attachment regions upstream and/or downstream of the construct described above, as set forth (for example) in U.S. Pat. Nos. 5,773,695 and 5,773,689 to Thompson et al.

The transgenic protein or peptide can be collected from the recombinant plant by any suitable technique, generally involving crushing or grinding plants or plant parts such as leaves, extracting the protein or peptide with a suitable solvent, and then isolating the protein or peptide with a purification technique such as chromatography, the choice of which will depend upon the particular protein or peptide being isolated. U.S. Pat. No. 6,037,456 to S. Garger et al. (Biosource Technologies Inc.) describes a method for obtaining a soluble protein or peptide from a plant comprising the sequential steps of: (a) homogenizing a plant to produce a green juice homogenate; (b) adjusting the pH of the green juice homogenate to less than or equal to about 5.2 (e.g., from about 4.0 to 5.2); (c) heating the green juice homogenate to a minimum temperature of about 45° C. (e.g., between about 45 and 50° C.); (d) centrifuging the green juice homogenate to produce a supernatant (and then optionally subjecting the supernatant to one or more ultrafiltration steps); and (e) purifying the protein or peptide from the supernatant (e.g., by chromatography, affinity-based method of purification, or salt precipitation). Examples of suitable proteins include, but are not limited to, those encoded by the transgenes mentioned above. It will be appreciated that that some proteins or peptides need not be isolated from the plant or plant parts, as where the protein or peptide is intended to be included as a feed ingredient with the plant.

The present invention is explained in greater detail in the following non-limiting Example.

### EXAMPLE 1

### Nicotine and Reducing Sugar Levels of Vector Burley 21-41 Compared to Parental Lines

The low nicotine tobacco variety, Vector Burley 21-41, was developed by transforming Burley 21 LA with the binary Agrobacterium vector, pYTY32, as described in international patent publication WO 98/56923, published under the Patent Cooperation Treaty. Burley 21 LA is a variety of Burley 21 with substantially reduced levels of nicotine as compared with Burley 21 (i.e., Burley 21 LA has 8% the nicotine levels of Burley 21, see Legg et al., (1971) Can. J. Genet. Cytol. 13:287-91; Legg et al., (1969) J. Hered. 60:213-17). Vector Burley 21-41 is most similar to the parent variety, Burley 21 LA. In general, Vector Burley 21-41 is similar to Burley 21 LA in all assessed characteristics, with the exception of alkaloid content (e.g., nicotine and nor-nicotine) and content of reducing sugars. Vector Burley 21-41 may be distinguished from the parent Burley 21 LA by its substantially reduced content of nicotine, nor-nicotine and total alkaloids. As shown below, total alkaloid concentrations in Vector Burley 21-41 are reduced to approximately 10% of the levels in the parent Burley 21 LA. Nicotine and nor-nicotine concentrations are less than approximately 6.7% and 32%, respectively, in Vector Burley 21-41 as compared with Burley 21 LA. Thus, Vector Burley 21-41 would have nicotine concentrations equivalent to 0.54% of the nicotine level of Burley 21. As shown in Table 1 below, it was unexpectedly found that Vector Burley 21-41 also has significantly higher (+87%) levels of reducing sugars as compared with Burley 21 LA (approximately 10.29% vs. 5.51%). Thus, it was found that reducing sugars in Vector Burley 21-41 are approximately 90% greater than in the parental line, making such plants much more desirable for use in applications such as described herein, particularly fermentative processes for the production of ethanol.

TABLE 1
Comparison between Vector Burley 21-41 and Burley 21 LA

| | TREATMENT | | |
|---|---|---|---|
| | Vector | Promoter | |
| | Burley 21-41 | Control | Burley 21 LA |
| Days from | 57.1 ± 3.6* | 56.7 ± 3.4* | 57.6 ± 3.4* |
| transplant to | | | |
| flowering (days) | | | |
| Height at flowering | 118.6 ± 20.1* | 112.1 ± 21.4* | 110.8 ± 19.5* |
| (cm) | | | |
| Yield (kg/ha) | 890.3 ± 70.7* | 780 ± 68.5* | 809.2 ± 71.2* |
| % Nicotine (× 10²) | 1.44 ± 0.66** | 19.12 ± 8.99* | 21.54 ± 9.34* |
| % Nor-Nicotine | 0.4 ± 0.1** | 1.56 ± 0.22* | 1.27 ± 0.52* |
| (× 10²) | | | |
| % Total Alkaloids | 0.23 ± 0.07** | 2.07 ± 0.93* | 2.31 ± 0.94* |
| % Total Nitrogen | 2.52 ± 0.78* | 2.96 ± 0.42* | 2.64 ± 0.91* |
| % Reducing Sugars | 10.29 ± 0.89** | 5.87 ± 2.04* | 5.51 ± 2.40* |

Data from 2000 field trial at Central Crops Research Station, Clayton, NC. Chemical analysis was carried out on topped plants. 15 replicates/10 plants per replicate. Data were analyzed using the F-test.
* = No significant difference,
** = Significant at the 1% level.

The foregoing is illustrative of the present invention, and is not to be construed as limiting thereof. The invention is defined by the following claims, with equivalents of the claims to be included therein.

What is claimed is:

1. A method for the fermentative production of ethanol, comprising:
providing a reduced nicotine recombinant tobacco plant or a portion thereof;
fermenting said plant or portion thereof in a fermentation vessel for a time sufficient to produce ethanol therefrom; and then
collecting said ethanol from said fermentation vessel.

2. A method according to claim 1, wherein said reduced nicotine recombinant tobacco plant exhibits reduced nicotine as compared to the parent plant from which said recombinant plant is produced.

3. A method according to claim 2, wherein said reduced nicotine recombinant tobacco plant or plant portion contains and expresses a heterologous nucleic acid that downregulates the production of nicotine in said recombinant plant.

4. A method according to claim 1, wherein said reduced nicotine recombinant tobacco plant or plant portion contains an increased amount of reducing sugars as compared to the parent plant from which said recombinant plant is produced.

5. A method according to claim 1, wherein said reduced nicotine recombinant tobacco plant or plant portion contains and expresses a heterologous DNA encoding at least a segment of an enzyme required for the biosynthesis of nicotine in tobacco, said recombinant plant exhibiting reduced levels of said enzyme as compared to a non-transformed control plant, reduced nicotine content as compared to a non-transformed control plant, and increased reducing sugars as compared to a non-transformed control plant.

6. The method according to claim 5, wherein said enzyme is selected from the group consisting of quinolate phosphoribosyl transferase, putrescine N-methyltransferase, arginine decarboxylase, omithine decarboxylase, S-adenosyl-methionine synthetase, NADH dehydrogenase, and phosphoribosylanthranilate isomerase.

7. The method according to claim 1, wherein said reduced nicotine recombinant tobacco plant or plant portion has reduced quinolate phosphoribosyl transferase (QPRTase) expression relative to a non-transformed control plant, said recombinant plant comprising recombinant plant cells containing:
an exogenous DNA construct comprising, in the 5′ to 3′ direction, a promoter operable in said plant cell and a heterologous DNA encoding at least a segment of a plant QPRTase mRNA, said heterologous DNA operably associated with said promoter, and with said heterologous DNA in sense or antisense orientation;
said recombinant plant exhibiting reduced QPRTase expression compared to a non-transformed control plant and reduced nicotine content as compared to a non-transformed control plant.

8. The method according to claim 1, wherein said reduced nicotine recombinant tobacco plant or plant portion has reduced putrescine N-methyltransferase (PMT) expression relative to a non-transformed control plant, said recombinant plant comprising recombinant plant cells containing:
an exogenous DNA construct comprising, in the 5′ to 3′ direction, a promoter operable in said plant cell and a heterologous DNA encoding at least a segment of a plant PMT mRNA, said heterologous DNA operably associated with said promoter, and with said heterologous DNA in sense or antisense orientation;
said recombinant plant exhibiting reduced PMT expression compared to a non-transformed control plant and reduced nicotine content as compared to a non-transformed control plant.

9. The method according to claim 1, wherein said plant or plant portion comprises cellulose.

10. The method according to claim 1, wherein said plant or plant portion comprises reducing sugars.

11. The method according to claim 1, further comprising the step of collecting a protein fraction from said plant or plant portion before or after said fermenting step.

12. A method of sustaining an animal subject, comprising:
feeding said animal subject reduced nicotine recombinant tobacco plant or a portion thereof.

13. A method according to claim 12, wherein said reduced nicotine recombinant tobacco plant or plant portion exhibits reduced nicotine as compared to the parent plant from which said recombinant plant is produced.

14. A method according to claim 13, wherein said reduced nicotine recombinant tobacco plant or plant portion contains and expresses a heterologous nucleic acid that downregulates the production of nicotine in said plant.

15. A method according to claim 12, wherein said reduced nicotine recombinant tobacco plant or plant portion contains an increased amount of reducing sugars as compared to the parent plant from which said recombinant plant is produced.

16. A method according to claim 12, wherein said reduced nicotine recombinant tobacco plant or plant portion contains and expresses a heterologous DNA encoding at least a segment of an enzyme required for the biosynthesis of nicotine in tobacco, said recombinant plant exhibiting reduced levels of said enzyme as compared to a non-transformed control plant, reduced nicotine content as compared to a non-transformed control plant, and increased reducing sugars as compared to a non-transformed control plant.

17. A method according to claim 12, wherein said feeding step is carried out by feeding said animal a forage crop comprising said reduced nicotine recombinant tobacco plant or plant portion.

18. A method according to claim 12, wherein said feeding step is carried out by feeding said animal leaves from said reduced nicotine recombinant tobacco plant or plant portion.

19. A method according to claim 12, wherein said feeding step is carried out by feeding said animal silage comprising said reduced nicotine recombinant tobacco plant or plant portion.

20. A method according to claim 12, wherein said feeding step is carried out by feeding said animal a food product comprising said reduced nicotine recombinant tobacco plant or plant portion in combination with at least one additional nutrient.

21. A food product comprising a reduced nicotine recombinant tobacco plant or a portion thereof.

22. A food product according to claim 21, wherein said reduced nicotine recombinant tobacco plant or plant portion exhibits reduced nicotine as compared to the parent plant from which said recombinant plant is produced.

23. A food product according to claim 22, wherein said reduced nicotine recombinant tobacco plant or plant portion contains and expresses a heterologous nucleic acid that downregulates the production of nicotine in said recombinant plant.

24. A method according to claim 21, wherein said reduced nicotine recombinant tobacco plant or plant portion contains an increased amount of reducing sugars as compared to the parent plant from which said recombinant plant is produced.

25. A method according to claim 21, wherein said reduced nicotine recombinant tobacco plant or plant portion contains and expresses a heterologous DNA encoding at least a segment of an enzyme required for the biosynthesis of nicotine in tobacco, said recombinant plant exhibiting reduced levels of said enzyme as compared to a non-transformed control plant, reduced nicotine content as compared to a non-transformed control plant, and increased reducing sugars as compared to a non-transformed control plant.

26. The food product according to claim 21, wherein said reduced nicotine recombinant tobacco plant or plant portion comprises tobacco leaves.

27. The food product according to claim 21, wherein said food product comprises silage.

28. The food product according to claim 21, wherein said food product comprises said reduced nicotine recombinant tobacco plant or plant portion in combination with at least one additional nutrient.

29. A method of producing a protein fraction from plant biomass, comprising the steps of:
providing reduced nicotine recombinant tobacco plant or a portion thereof; and then
collecting a protein fraction from said recombinant plant or plant portion.

30. A method according to claim 29, wherein said reduced nicotine recombinant tobacco plant or plant portion exhibits reduced nicotine as compared to the parent plant from which said recombinant plant is produced.

31. A method according to claim 30, wherein said reduced nicotine recombinant tobacco plant or plant portion contains and expresses a heterologous nucleic acid that downregulates the production of nicotine in said recombinant plant.

32. A method according to claim 29, wherein said reduced nicotine recombinant tobacco plant or plant portion contains an increased amount of reducing sugars as compared to the parent plant from which said recombinant plant is produced.

33. A method according to claim 29, wherein said reduced nicotine recombinant tobacco plant or plant portion contains and expresses a heterologous DNA encoding at least a segment of an enzyme required for the biosynthesis of nicotine in tobacco, said recombinant plant exhibiting reduced levels of said enzyme as compared to a non-transformed control plant, reduced nicotine content as compared to a non-transformed control plant, and increased reducing sugars as compared to a non-transformed control plant.

34. The method of claim 29, wherein said protein fraction comprises fraction I protein.

35. The method of claim 29, wherein said protein fraction consists essentially of fraction I protein.

36. The method of claim 29, wherein said protein fraction comprises fraction II proteins.

37. The method of claim 29, wherein said protein fraction consists essentially of fraction II proteins.
